SEQUENCE LISTING (7 sequences)

SEQ ID NO: 1 - protein, 9 aa, Artificial Sequence
  synthesized peptide
QAFEPIRSV

SEQ ID NO: 2 - protein, 12 aa, Artificial Sequence
  synthesized peptide
TNPWHSPRQGSF

SEQ ID NO: 3 - protein, 4 aa, Artificial Sequence
  synthesized peptide
QAFE

SEQ ID NO: 4 - protein, 5 aa, Artificial Sequence
  synthesized peptide
PIRSV

SEQ ID NO: 5 - protein, 4 aa, Artificial Sequence
  synthesized peptide
TNPW

SEQ ID NO: 6 - protein, 4 aa, Artificial Sequence
  synthesized peptide
HSPR

SEQ ID NO: 7 - protein, 4 aa, Artificial Sequence
  synthesized peptide
QGSF

(19) United States
(12) Patent Application Publication
(10) Pub. No.: US 2024/0287132 A1
(43) Pub. Date: Aug. 29, 2024

(54) PEPTIDE, COMPOSITION, AND GHRELIN SECRETION PROMOTING AGENT

(71) Applicants: KYOTO UNIVERSITY, Kyoto (JP); Kazusa DNA Research Institute, Chiba (JP); KAMEDA SEIKA CO., LTD., Niigata (JP)

(72) Inventors: Kousaku Ohinata, Kyoto (JP); Yuki Tokuyama, Kyoto (JP); Junya Nakato, Kyoto (JP); Hiroshi Iwakura, Kyoto (JP); Kentaro Kaneko, Kyoto (JP); Sayano Odaka, Kyoto (JP); Masaru Sato, Chiba (JP); Hideyuki Suzuki, Chiba (JP); Akira Ito, Niigata (JP); Yuki Higuchi, Niigata (JP); Ryoko Nakayama, Niigata (JP)

(21) Appl. No.: 17/605,811

(22) PCT Filed: Apr. 23, 2020
(86) PCT No.: PCT/JP2020/017565

§ 371 Date: Oct. 22, 2021

(30) Foreign Application Priority Data
Apr. 26, 2019 (JP) 2019-086211

(51) Int. Cl. C07K 7/06; A61K 38/00; A61P 1/14; C07K 5/10; C07K 7/08

(52) U.S. Cl. CPC C07K 7/06; A61P 1/14; C07K 5/10; C07K 7/08; A61K 38/00

(57) ABSTRACT

The present invention addresses the problem of providing a novel peptide which can treat, prevent or ameliorate anorexia. The present invention provides a peptide described in any one of the following items (1) to (3). (1) A peptide consisting of 4 to 9 inclusive contiguous amino acid residues in the amino acid sequence represented by SEQ ID NO: 1; (2) a peptide consisting of 4 to 12 inclusive contiguous amino acid residues in the amino acid sequence represented by SEQ ID NO: 2; and (3) a peptide consisting of an amino acid sequence having 90% or higher identity to the amino acid sequence represented by SEQ ID NO: 2.

### TECHNICAL FIELD

The present invention relates to peptides, a composition, and a ghrelin secretion promoting agent.

### BACKGROUND ART

Anorexia is known as one of the symptoms caused by various diseases and stress. If people suffer from anorexia for a long time, they may have poor nutrition and serious illness. Even with no illness, elderly people are found to have anorexia, which may degrade their quality of life (QOL).

For example, Patent Document 1 discloses a ghrelin secretion promoting agent that is for preventing and treating ghrelin secretion related symptoms, such as anorexia, and comprises a specific compound.

Patent Document 1: Japanese Unexamined Patent Application, Publication No. 2013-227309

### DISCLOSURE OF THE INVENTION

### Problems to be Solved by the Invention

However, a further need exists for functional materials available for treating, preventing, or ameliorating anorexia.

The present invention has been made in light of the circumstances described above, and it is an object of the present invention to provide a novel peptide available for treating, preventing, or ameliorating anorexia.

### Means for Solving the Problems

The present inventors have completed the present invention based on findings that the problem can be solved using a peptide consisting of a specific amino acid sequence. Specifically, the present invention provides the following aspects.

<1> A peptide selected from the group consisting of:
(1) a peptide consisting of 4 or more and 9 or less contiguous amino acid residues of the amino acid sequence set forth in SEQ
(2) a peptide consisting of 4 or more and 12 or less contiguous amino acid residues of the amino acid sequence set forth in SEQ ID NO: 2; and ID NO: 1;
(3) a peptide consisting of an amino acid sequence having 90% or more identity to the amino acid sequence set forth in SEQ ID NO: 2.

<2> The peptide according to aspect <1>, consisting of the amino acid sequence set forth in any one of SEQ ID NOS: 3 to 7.

<3> The peptide according to aspect <1>, consisting of the amino acid sequence set forth in SEQ ID NO: 1 or 2 and being a subtilisin digest.

<4> A composition for use in treating, preventing, or ameliorating anorexia, the composition comprising the peptide according to any one of aspects <1> to <3> or a protein comprising an amino acid sequence comprising, in part, the amino acid sequence of the peptide.

<5> The composition according to aspect <4>, wherein the anorexia is caused by a decrease in ghrelin secretion.

<6> The composition according to aspect <4> or <5>, being a pharmaceutical composition.

<7> The composition according to aspect <4> or <5>, being a food or drink product.

<8> A method of treating, preventing, or ameliorating anorexia, the method comprising administering the composition according to any one of aspects <4> to <7>.

<9> A ghrelin secretion promoting agent, comprising a peptide selected from the group consisting of:
(1) a peptide consisting of 4 or more and 9 or less contiguous amino acid residues of the amino acid sequence set forth in SEQ ID NO: 1;
(2) a peptide consisting of 4 or more and 12 or less contiguous amino acid residues of the amino acid sequence set forth in SEQ ID NO: 2; and
(3) a peptide consisting of an amino acid sequence having 90% or more identity to the amino acid sequence set forth in SEQ ID NO: 2.

### Effects of the Invention

The present invention provides novel peptides available for treating, preventing, or ameliorating anorexia.

### BRIEF DESCRIPTION OF THE DRAWINGS

FIG. 1 is a graph showing the effect of enzymatic digests of rice endosperm protein on ghrelin secretion activity;

FIG. 2 is a graph showing the effect of peptides according to the present invention on ghrelin secretion activity;

FIGS. 3A, 3B, 3C, and 3D are graph showing the effect of a peptide according to the present invention on ghrelin secretion activity and ghrelin synthesis-related gene expression;

FIG. 4 is a graph showing the effect of a peptide according to the present invention on intracellular CAMP level;

FIGS. 5A and 5B are graphs showing the effect of a peptide according to the present invention on the eating behavior of mice and serum ghrelin level; and

FIG. 6 is a graph showing the effect of peptides according to the present invention on the eating behavior of mice.

### PREFERRED MODE FOR CARRYING OUT THE INVENTION

Hereinafter, embodiments of the preset invention will be described in detail. It will be understood that the embodiments described below are not intended to limit the present invention.

### <Peptides According to the Invention>

The present invention is directed to a peptide selected from peptides (1), (2), and (3) shown below. Hereinafter, amino acid sequences are shown from their N-terminus at the left end to their C-terminus.
(1) A peptide consisting of 4 or more and 9 or less contiguous amino acid residues of the amino acid sequence (QAFEPIRSV) set forth in SEQ ID NO: 1.
(2) A peptide consisting of 4 or more and 12 or less contiguous amino acid residues of the amino acid sequence (TNPWHSPROGSF) set forth in SEQ ID NO: 2.
(3) A peptide consisting of an amino acid sequence having 90% or more identity to the amino acid sequence set forth in SEQ ID NO: 2.

Regarding the present invention, the expression “a peptide consisting of n contiguous amino acid residues of the amino acid sequence set forth in SEQ ID NO: 1 (or 2)” means a peptide consisting of n amino acid residues selected from the amino acid sequence set forth in SEQ ID NO: 1 (or 2) with their sequence remaining unchanged. For example, the expression “a peptide consisting of 4 contiguous amino acid residuals of the amino acid sequence set forth in SEQ ID No: 1” include the peptide “QAFE”, the peptide “AFEP”, and the peptide “FEPI”.

The present inventors have conducted studies based on information regarding simultaneous analysis of a mixture of various peptides and information regarding the correlation between known peptide structures having an effect on anorexia and their activity. As a result, the present inventors have found novel peptides effective for the treatment or the like of anorexia, specifically, the peptides shown above. The amino acid sequences set forth in SEQ ID NOS: 1 and 2 are all enzymatic digests of rice endosperm protein, which are produced using subtilisin (also called “sachilysin”).

The peptide of the present invention may be a peptide consisting of the amino acid sequence set forth in SEQ ID NO: 1 or 2 or a peptide consisting of a part of the amino acid sequence set forth in SEQ ID NO: 1 or 2.

When the peptide of the present invention is a peptide consisting of a part of the amino acid sequence set forth in SEQ ID NO: 1, the peptide preferably consists of 4 or more and 8 or less contiguous amino acid residues and more preferably consists of 4 or more and 5 or less contiguous amino acid residues of the amino acid sequence set forth in SEQ ID NO: 1.

When the peptide of the present invention is a peptide consisting of a part of the amino acid sequence set forth in SEQ ID NO: 2, the peptide preferably consists of 4 or more and 10 or less contiguous amino acid residues and more preferably consists of 4 or more and 8 or less contiguous amino acid residues of the amino acid sequence set forth in SEQ ID NO: 2.

When the peptide of the present invention is a peptide consisting of a part of the amino acid sequence set forth in SEQ ID NO: 1, preferred sequences of the peptide include the following:
A peptide consisting of the amino acid sequence (QAFE) set forth in SEQ ID NO: 3; and
A peptide consisting of the amino acid sequence (PIRSV) set forth in SEQ ID NO: 4.

When the peptide of the present invention is a peptide consisting of a part of the amino acid sequence set forth in SEQ ID NO: 2, preferred sequences of the peptide include the following:
A peptide consisting of the amino acid sequence (TNPW) set forth in SEQ ID NO: 5;
A peptide consisting of the amino acid sequence (HSPR) set forth in SEQ ID NO: 6; and
A peptide consisting of the amino acid sequence (QGSF) set forth in SEQ ID NO: 7.

The peptide of the present invention may be a subtilisin digest consisting of the amino acid sequence set forth in SEQ ID NO: 1 or 2.

As a result of further studies, the present inventors have found that the peptide of the present invention is effective in promoting ghrelin secretion to increase the appetite and effective for treating or preventing anorexia.

The peptide of the present invention may be obtained by chemical synthesis or hydrolysis of naturally occurring proteins (rice endosperm protein) or polypeptides.

Examples of the chemical synthesis method include known peptide synthesis methods. Specific examples thereof include liquid phase methods or solid phase methods, which are commonly used for peptide synthesis. More specifically, Fmoc methods, Boc methods, and so on may be used. The synthesized peptides may be purified. Examples of the purification method include methods using ion exchange chromatography, reverse phase liquid chromatography, or affinity chromatography.

Examples of the hydrolysis method include methods using a hydrolytic enzyme, a strong acid, or a strong base.

The hydrolytic enzyme method may be carried out using an animal-, plant-, or microorganism-derived hydrolytic enzyme (such as subtilisin). Microorganisms acceptable for food production (e.g., food yeasts, such as baker's yeasts and beer yeasts) may also be used as substitute for the hydrolytic enzyme.

The hydrolysis using the hydrolytic enzyme may be carried out under any conditions and may include adjusting pH to a suitable level depending on the enzyme used; and carrying out the reaction at a temperature of about 30 to about 40° C. for 30 minutes to 48 hours. The peptide of the present invention may be purified from the resulting reaction solution before use. When a food material is hydrolyzed, the resulting hydrolysis product may be directly used as a food product or may be added to any other food material to form a food product.

The strong acid method may be carried out using, for example, hydrochloric acid, nitric acid, or sulfuric acid. The strong base method may be carried out using, for example, an alkali metal hydroxide (e.g., sodium hydroxide, potassium hydroxide, lithium hydroxide), an alkali metal carbonate (e.g., sodium carbonate, potassium carbonate), or an alkali metal hydrogencarbonate (e.g., sodium hydrogencarbonate, potassium hydrogencarbonate).

The hydrolysis using the strong acid or strong base may be carried out under any conditions and may include carrying out the reaction in water at a temperature of 1 to 100° C. for 30 minutes to 48 hours in the presence of the strong acid or strong base. The hydrolysis reaction product may be directly used after being adjusted for pH or may be subjected to purification to isolate the peptide of the present invention before use.

The amino acid sequence of the peptides obtained by the various methods can be analyzed using GC-MS, a protein sequencer that reads the amino acid sequence from the C-terminus by using Edman degradation technique, or the like.

### <Composition of the Invention>

The present invention is directed to a composition comprising at least the peptide of the present invention. The composition of the present invention may consist of the peptide of the present invention and optionally an additional component. In the composition of the present invention, the peptide may be a part of a protein, which includes the peptide of the present invention as a part of its amino acid sequence.

The peptide of the present invention may be taken to treat, prevent, or ameliorate anorexia. Therefore, the composition of the present invention is preferably used to treat, prevent, or ameliorate anorexia.

Regarding the present invention, the term “anorexia” means loss of appetite or decline in dietary intake. In particular, the present invention makes it possible to successfully treat, prevent, or ameliorate anorexia caused by a decrease in ghrelin secretion.

Regarding the present invention, the term “treat” means, for example, to heal anorexia. The term “prevent” means, for example, to reduce or slow the development of anorexia. The term “ameliorate” means, for example, to alleviate or reduce anorexia.

The composition of the present invention may be prepared in any form and may be prepared as a pharmaceutical composition or a food or drink product.

When the composition of the present invention is prepared as a pharmaceutical composition, it may be prepared as an oral administration agent or a parenteral administration agent. The composition of the present invention may be prepared, for example, using the peptide of the present invention alone or in combination with a carrier, a diluent, or an excipient, in the form of any of the following formulations: a tablet (e.g., uncoated tablet, sugar-coated tablet, forming tablet, film-coated tablet, chewable tablet), a capsule, a lozenge, a powder, fine granules, granules, a solution, a suspension, an emulsion, a paste, a cream, an injection (including a formulation to be added to an infusion solution such as an amino acid infusion or an electrolyte infusion), an enteric-coated tablet or capsule, and a sustained release formulation.

The carrier, diluent, or excipient may include a material that is commonly used in the field of drug formulation and inert to the peptide of the present invention. Examples of such a material include lactose, glucose, mannitol, dextrin, cyclodextrin, starch, sucrose, magnesium aluminate metasilicate, synthetic aluminum silicate, sodium carboxymethylcellulose, hydroxypropyl starch, calcium carboxymethylcellulose, ion exchange resin, methylcellulose, gelatin, gum arabic, hydroxypropylcellulose, hydroxypropylmethylcellulose, polyvinylpyrrolidone, polyvinyl alcohol, light anhydrous silicic acid, magnesium stearate, talc, tragacanth, bentonite, Veegum, titanium oxide, sorbitan fatty acid ester, sodium lauryl sulfate, glycerin, glycerin fatty acid esters, purified lanolin, glycerogelatin, polysorbate, macrogol, vegetable oil, wax, liquid paraffin, white petrolatum, fluorocarbon, nonionic surfactants, propylene glycol, and water.

When the composition of the present invention is prepared as a food or drink product, it may be prepared in any form, such as a beverage (e.g., (coffee, cocoa, juice, soft drink, mineral beverage, tea beverage, green tea, black tea, oolong tea, milk beverage, lactic acid bacteria beverage, yogurt beverage, carbonated beverage), a gum, a gummy candy, a jelly, a candy, a cookie, a cracker, a biscuit, ice confectionery (e.g., ice cream, ice candy, sherbet, shaved ice), a retort food product, or a jelly food product (e.g., jelly, agar, jelly beverage).

The food or drink product of the present invention may be prepared as what is called a health food product, a functional food product, a dietary supplement, a supplement, a food for specified health use, a food with a functional claim, a food or food combination for sick people (a type of food product for specialized use defined by the Ministry of Health, Labour and Welfare of Japan), or a food for elderly people (a type of food product for specialized use defined by the Ministry of Health, Labour and Welfare of Japan).

The content of the peptide of the present invention in the composition of the present invention may be set as appropriate depending on the effect to be obtained or other conditions. For example, the composition preferably contains 0.01% by mass or more, more preferably 1.00% by mass or more of the peptide of the present invention. The composition also preferably contains 100% by or less, more preferably 90% by mass of less of the peptide of the present invention. The composition of the present invention may contain other peptides than the peptide of the present invention. In such a case, the content shown above is calculated as the content of the peptide of the present invention.

The content of an additional component other than the peptide of the present invention in the composition of the present invention may be set as appropriate depending on the type of the additional component, the form of the composition, the effect to be obtained, or other conditions.

The composition of the present invention may be administered by any method. The administration may be oral administration or parenteral administration (e.g., injection). Preferably, the composition of the present invention is orally administered so that the advantageous effect of the present invention can be easily brought about.

For example, the composition of the present invention is preferably administered at a dose of 0.01 mg/kg to 500 mg/kg, more favorably 0.05 mg/kg to 100 mg/kg, even more favorably 0.1 mg/kg to 30 mg/kg, per day to an adult person, which is calculated in terms of the amount of the peptide of the present invention, although it depends on the administration method and the condition, age, and other factors of the administration target. As the dosage increases within the range shown above, the advantageous effect of the present invention tends to be easily produced.

The composition of the present invention may be produced using a known method, which is selected depending on the formulation to be obtained.

### <Method of Treating, Preventing, or Ameliorating Anorexia>

The composition of the present invention may be administered to a subject to treat, prevent, or ameliorate anorexia.

The administration method may be appropriately selected depending on the formulation of the composition.

The number of times of administration, the intervals of administration, and the dosage may be appropriately selected depending on the conditions (e.g., symptom, age, weight) of the subject of administration.

Examples of the subject of administration include, but are not limited to, humans, non-human mammals (e.g., dogs, cats, domestic animals (e.g., cattle, pigs, sheep, goats)), and so on.

### <Ghrelin Secretion Promoting Agent>

As described above, the peptide of the present invention has a ghrelin secretion promoting effect. Thus, the peptide of the present invention is effective as a ghrelin secretion promoting agent.

The ghrelin secretion promoting agent of the present invention includes at least the peptide of the present invention, and may consist of the peptide of the present invention or may comprise the peptide of the present invention and an additional component. In the ghrelin secretion promoting agent of the present invention, the additional component may be any component having no inhibitory effect on the activity of the peptide of the present invention. For example, the ghrelin secretion promoting agent of the present invention may contain any of the components shown above in the section <Composition of the Invention>.

### EXAMPLES

Hereinafter, the present invention will be specifically described with reference to examples, which are not intended to limit the present invention.

### Test 1

### (Peptide Preparation 1)

Purified rice endosperm protein and one of the different digestive enzymes were dissolved in water to form a mixture with a mass ratio of rice endosperm protein to enzyme of 100:1 and a rice endosperm protein final concentration of 20 mg/ml. The mixture underwent the enzymatic reaction to give peptides as enzymatic digests.

The digestive enzymes used and the reaction conditions are as follows. After the elapse of the reaction time shown below, each sample was boiled (at 100° C. for 10 minutes) so that the enzymatic reaction was quenched.
(1) For subtilisin (T1426 (trade name) manufactured by Sigma-Aldrich), the reaction was carried out at a temperature of 37° C. and a pH of 7.5 for a time period of 5 hours.
(2) For Sumizyme (Sumizyme FP (trade name) manufactured by Shinnihon Chemicals Corporation), the reaction was carried out at a temperature of 50° C. and a pH of 7.5 for a time period of 5 hours.
(3) For Thermolysin (Thermolysin (trade name) manufactured by Seikagaku Corporation), the reaction was carried out at a temperature of 37° C. and a pH of 7.0 to 7.5 for a time period of 5 hours.

### (Ghrelin Secretion Activity Evaluation 1)

The peptides obtained as shown above (Peptide Preparation 1) were subjected to the test shown below for evaluating the effect of each peptide on ghrelin secretion. More promotion of ghrelin secretion by peptide administration means that the peptide will have a higher appetite-enhancing effect.

Ghrelin-secreting cells (MGN3-1) were seeded at 1×10⁵ cells/well onto a 96-well plate and cultured in a sodium octanoate/DMEM medium at 37° C. for 24 hours. After the cultivation, the cells were washed with DPBS. A buffer solution of each peptide was prepared and, 100 μL of the buffer solution (with a peptide concentration of 1 mg/mL or 10 mg/mL) was added to the cells. The cells were further cultured at 37° C. for 4 hours. The buffer used contained 50 μM of sodium octanoate in DMEM.

Unless otherwise stated, all tests shown below were performed using, as a control, a culture obtained in the same manner as shown above except that only the buffer was added instead of the buffer solution of the peptide.

After the cultivation, the medium sample was collected and centrifuged to give a supernatant. Subsequently, 10 μL of 1 N HCl was added to the supernatant, and the resulting sample was stored at −80° C. until it was subjected to the ghrelin concentration measurement shown below.

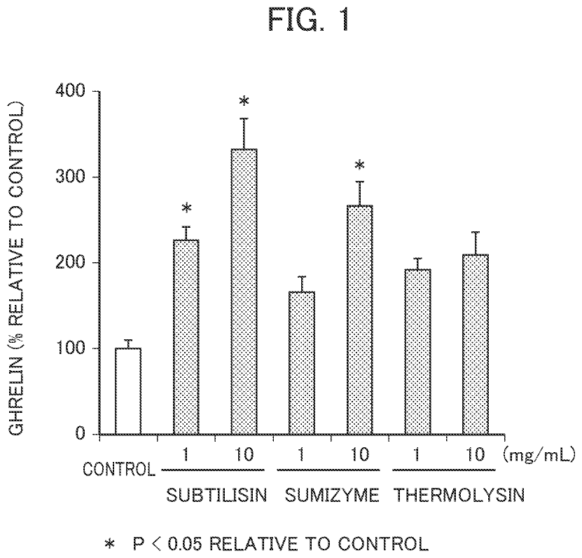

The concentration of ghrelin in each medium sample was measured using an ELISA kit (Ghrelin (Acylated) EIA Kit A05117 (trade name) manufactured by Bertin Pharma SAS). The results are shown in FIG. 1. The results shown in FIG. 1 are values relative to the measured value of the control.

As shown in FIG. 1, the subtilisin digest, the Sumizyme digest, and the Thermolysin digest were found to each have a ghrelin secretion promoting effect. In particular, the subtilisin digest and the Sumizyme digest were found to have a significant, concentration-dependent, ghrelin secretion promoting effect.

### Test 2

### (Peptide Preparation 2)

Based on the results shown above (Ghrelin Secretion Activity Evaluation 1), the peptides in the subtilisin digest, which had a ghrelin secretion promoting effect, were screened, and as a result, the two candidate peptides shown below were identified. Afterward, these peptides were synthesized by Fmoc method and then purified by reversed-phase HPLC. It should be noted that hereinafter, a “peptide consisting of the amino acid sequence set forth in SEQ ID NO: n” is also simply referred to as “a peptide of SEQ ID NO: n”.
(1) A peptide consisting of the amino acid sequence (QAFEPIRSV) set forth in SEQ ID NO: 1
(2) A peptide consisting of the amino acid sequence (TNPWHSPROGSF) set forth in SEQ ID NO: 2

### (Ghrelin Secretion Activity Evaluation 2)

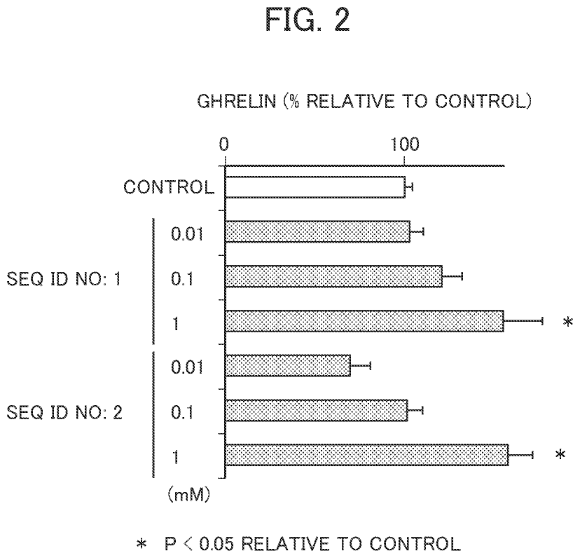

The two peptides were evaluated for their effect on ghrelin secretion by the same method as shown above (Ghrelin Secretion Activity Evaluation 1). The results are shown in FIG. 2 (n=4). The peptide solution used in this example had a peptide concentration of 0.01, 0.1, or 1 mM.

As shown in FIG. 2, both the peptides of SEQ ID NOS: 1 and 2 were found to have a ghrelin secretion promoting effect. In particular, the effect was significant at a peptide concentration of 1 mM or more.

### Test 3

The peptide of SEQ ID NO: 1 prepared as shown above (Peptide Preparation 2) was verified for its ghrelin secretion promoting effect. Specifically, the administration of the peptide of SEQ ID NO: 1 was verified for its effect on intracellular and extracellular ghrelin levels and for its effect on ghrelin synthesis-related gene expression.

### (Ghrelin Secretion Activity Evaluation 3)

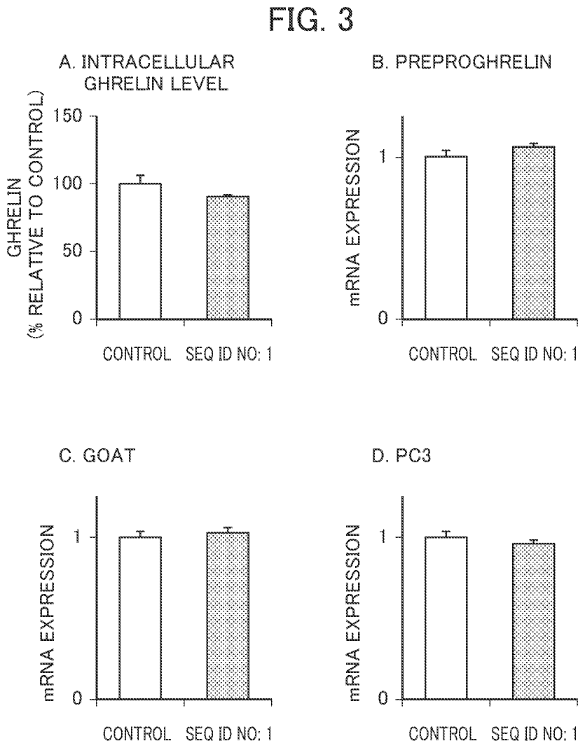

The peptide of SEQ ID NO: 1 was evaluated for its effect on ghrelin secretion (intracellular ghrelin level) by the same method as shown above (Ghrelin Secretion Activity Evaluation 1). The results are shown in FIG. 3A (n=4 or 5).

### (Evaluation of Ghrelin Synthesis-Related Gene Expression)

The expression levels of the three, ghrelin synthesis-related genes (Preproghrelin, GOAT, and PC3) in the cells obtained after the completion of the cultivation shown above (Ghrelin Secretion Activity Evaluation 1) were measured by the method shown below. First, mRNA was extracted from the cells using RNeasy Mini Kit (manufactured by QIA-GEN) and Takara PrimeScript RT Master Mix (manufactured by Takara Bio Inc.). Subsequently, the mRNA expression level was measured through the amplification of cDNA using LightCycler 96 System (manufactured by Roche Diagnostics K.K.) and THUNDERBIRD qPCR Mix (manufactured by Toyobo Co., Ltd.). The results are shown in FIG. 3B (Preproghrelin), FIG. 3C (GOAT), and FIG. 3D (PC3) (n=4 or 5).

As shown in FIG. 3A, the administration of the peptide of SEQ ID NO: 1 had a tendency to reduce the intracellular ghrelin level. The administration of the peptide of SEQ ID NO: 1 increased the extracellular ghrelin level (data not shown).

As shown in FIGS. 3B, 3C, and 3D, the administration of the peptide of SEQ ID NO: 1 was not found to change any of the expression levels of the ghrelin synthesis-related genes.

The results shown above demonstrated that the peptide of SEQ ID NO: 1 produced a ghrelin secretion promoting effect by promoting extracellular secretion of ghrelin not by promoting ghrelin synthesis.

### Test 4

Based on the results of <Test 3> shown above, the peptide of SEQ ID NO: 1 was verified for its ghrelin secretion promoting effect. Specifically, the administration of the peptide of SEQ ID NO: 1 was verified for its effect on intracellular CAMP level and intracellular calcium ion (Ca²⁺) level.

In this regard, noradrenaline is known to promote ghrelin secretion by increasing the intracellular CAMP level and the intracellular calcium ion level. In the test shown below, therefore, noradrenaline was also used to provide a positive control.

### (Evaluation of Intracellular CAMP Level)

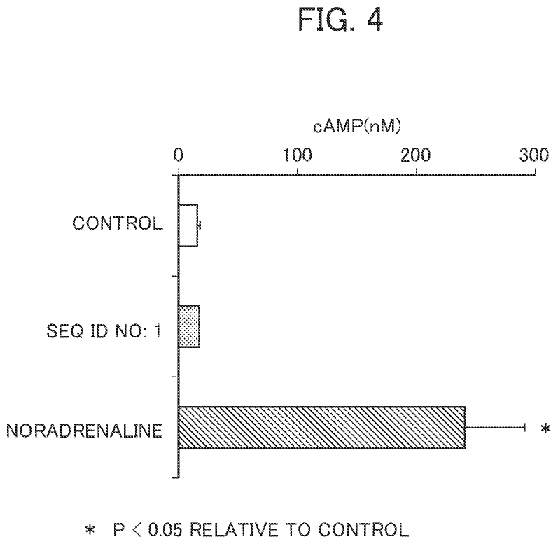

Ghrelin-secreting cells (MGN3-1) were lysed in a Krebs-Ringer-HEPES buffer containing 0.5 mM IBMX. After the mixture was incubated at 37° C. for 30 minutes, the intracellular CAMP was quantified using HitHunter CAMP Assay for Small Molecules (manufactured by DiscoveRx). The results are shown in FIG. 4.

### (Evaluation of Intracellular Calcium Ion Level)

The intracellular calcium ion level was measured using Calcium Kit II-Fluo 4 (Dojindo Laboratories) and a multi-plate reader (manufactured by BMG LABTECH GmbH, Aex: 485 nm, Xem: 520 nm).

As shown in FIG. 4, the peptide of SEQ ID NO: 1 had no effect on the intracellular CAMP level. The peptide of SEQ ID NO: 1 also had no effect on the intracellular calcium ion level (data not shown). This suggests that the peptide of SEQ ID NO: 1 should promote ghrelin secretion through a pathway different from the noradrenaline pathway.

### Test 5

The peptide of SEQ ID NO: 1 was administered to mice and verified for its in vivo effect. Specifically, the administration of the peptide of SEQ ID NO: 1 was verified for its effect on the eating behavior of mice and on serum ghrelin level.

### (Evaluation 1 of Eating Behavior of Mice)

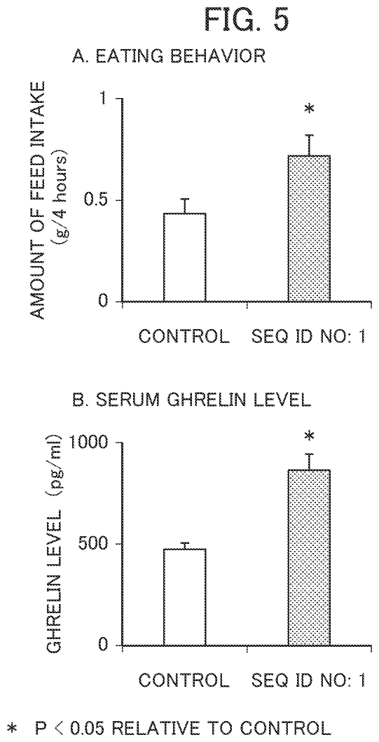

Mice (11 weeks old male ddY mice) were orally administered with a single dose of a saline solution of the peptide of SEQ ID NO: 1 (1 mg/kg). Subsequently, a previously weighed feed (CE-2 (trade name) manufactured by CLEA Japan, Inc.) was given to the mice, and 4 hours after the feeding, the weight of the remaining feed was measured. A control test was also carried out in which the same procedure was carried out as shown above except that a single dose of saline was orally administered instead of the peptide of SEQ ID NO: 1. The amount of feed intake (g/4 hours) was defined as the difference between the weight of the feed given to the mice and the weight of the feed remaining 4 hours after the feeding. The results are shown in FIG. 5A (n=10 or 11).

### (Evaluation of Mouse Serum Ghrelin Level)

Mice (11 weeks old male ddY mice) were administered with a saline solution of the peptide of SEQ ID NO: 1 (0.3 mg/kg). Subsequently, 1 hour after the administration, blood was collected from the orbital vein of the mice and centrifuged at 4° C. at 1,120×g for 10 minutes. The resulting supernatant was acidified with 1 N HCl and then measured for serum ghrelin level (pg/ml) using an acylated ghrelin enzyme immunoassay kit. The results are shown in FIG. 5B (n=5 to 7).

As shown in FIG. 5A, the peptide of SEQ ID NO: 1 increased the amount of feed intake by the mice and had an appetite enhancing effect. Moreover, as shown in FIG. 5B, the administration of the peptide of SEQ ID NO: 1 increased the serum ghrelin level.

### Test 6

The peptides of SEQ ID NOS: 1 and 2 prepared as shown above (Peptide Preparation 2) and partial peptides of them (the peptides of SEQ ID NOS: 3 to 7) were synthesized by Fmoc method and then purified by reversed-phase HPLC. Each of these peptides was administered to mice and verified for its in vivo effect. Specifically, the administration of each of the peptides was verified for its effect on the eating behavior of mice.

The peptides used in this example were the following seven peptides:
(1) A peptide consisting of the amino acid sequence (QAFEPIRSV) set forth in SEQ ID NO: 1;
(2) A peptide consisting of the amino acid sequence (TNPWHSPROGSF) set forth in SEQ ID NO: 2;
(3) A peptide consisting of the amino acid sequence (QAFE) set forth in SEQ ID NO: 3;
(4) A peptide consisting of the amino acid sequence (PIRSV) set forth in SEQ ID NO: 4;
(5) A peptide consisting of the amino acid sequence (TNPW) set forth in SEQ ID NO: 5;
(6) A peptide consisting of the amino acid sequence (HSPR) set forth in SEQ ID NO: 6; and
(7) A peptide consisting of the amino acid sequence (QGSF) set forth in SEQ ID NO: 7.

### (Evaluation 2 of Eating Behavior of Mice)

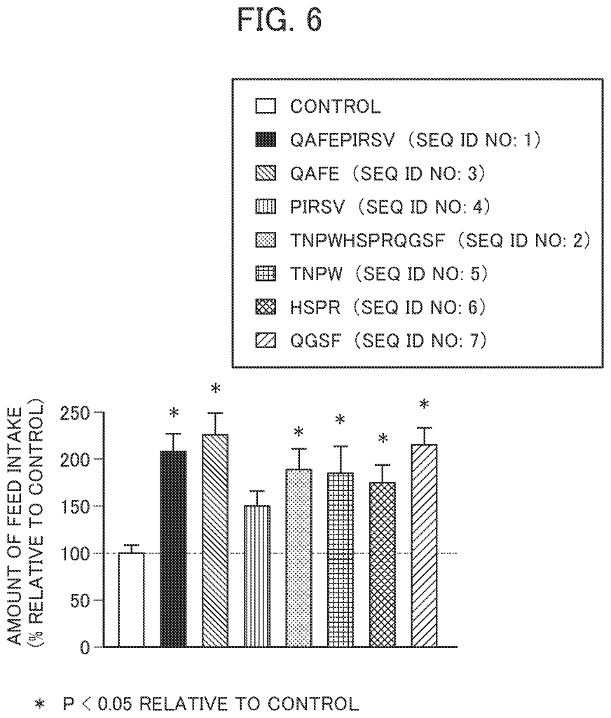

Mice (7 weeks old male ddY mice acclimated for at least 1 week before use after being purchased) were orally administered with a single dose of a saline solution of each peptide (1 mg/kg). Subsequently, a previously weighed feed (CE-2 (trade name) manufactured by CLEA Japan, Inc.) was given to the mice, and 2 hours after the feeding, the weight of the remaining feed was measured. A control test was also carried out in which the same procedure was carried out as shown above except that a single dose of saline was orally administered instead of each peptide. The amount of feed intake (g/2 hours) was defined as the difference between the weight of the feed given to the mice and the weight of the feed remaining 2 hours after the feeding. Based on the results, the amount of feed intake in the control group was normalized to 100, and the amount of feed intake in each group was calculated relative to 100. The results are shown in FIG. 6 (n=7 to 18, *: P<0.05 relative to the control).

As shown in FIG. 6, all the peptides increased the amount of feed intake by the mice and had an appetite enhancing effect. Not only the peptide consisting of the amino acid sequence set forth in SEQ ID NO: 1 or 2 but also partial peptides of the peptide of SEQ ID NO: 1 or 2 were found to have such an effect. In particular, the peptide consisting of the amino acid sequence set forth in SEQ ID NO: 3, 5, 6, or 7 significantly increased the amount of feed intake as compared to the control.

1. A peptide selected from the group consisting of:
(1) a peptide consisting of 4 or more and 9 or less contiguous amino acid residues of an amino acid sequence set forth in SEQ ID NO: 1;
(2) a peptide consisting of 4 or more and 12 or less contiguous amino acid residues of an amino acid sequence set forth in SEQ ID NO: 2; and
(3) a peptide consisting of an amino acid sequence having 90% or more identity to the amino acid sequence set forth in SEQ ID NO: 2.

2. The peptide according to claim 1, consisting of an amino acid sequence set forth in any one of SEQ ID NOS: 3 to 7.

3. The peptide according to claim 1, consisting of an amino acid sequence set forth in SEQ ID NO: 1 or 2 and being a subtilisin digest.

4. A composition for use in treating, preventing, or ameliorating anorexia, the composition comprising the peptide according to claim 1 or a protein comprising an amino acid sequence comprising, in part, the amino acid sequence of the peptide.

5. The composition according to claim 4, wherein the anorexia is caused by a decrease in ghrelin secretion.

6. The composition according to claim 4, being a pharmaceutical composition.

7. The composition according to claim 4, being a food or drink product.

8. A method of treating, preventing, or ameliorating anorexia, the method comprising administering the composition according to claim 4.

9. A ghrelin secretion promoting agent, comprising a peptide selected from the group consisting of:
(1) a peptide consisting of 4 or more and 9 or less contiguous amino acid residues of an amino acid sequence set forth in SEQ ID NO: 1;
(2) a peptide consisting of 4 or more and 12 or less contiguous amino acid residues of an amino acid sequence set forth in SEQ ID NO: 2; and
(3) a peptide consisting of an amino acid sequence having 90% or more identity to the amino acid sequence set forth in SEQ ID NO: 2.
